(19) United States
(12) Patent Application Publication
(10) Pub. No.: US 2004/0092987 A1
(43) Pub. Date: May 13, 2004

(54) Surgical device for removal of tissue cells from

(76) Inventor: Andreas Pein, Gross Gronau (DE)

Correspondence Address: Horst M Kasper, 13 Forest Drive, Warren, NJ, 07059, US

(21) Appl. No.: 10/332,524

(22) PCT Filed: Jul. 5, 2001
(86) PCT No.: PCT/DE01/02417

(30) Foreign Application Priority Data
Jul. 7, 2000 (DE) 100 33 278.1

(51) Int. Cl. A61B 17/32

(52) U.S. Cl. 606/167

(57) ABSTRACT

Fatty tissue is conventionally removed from the human body by disrupting the fatty tissue cells, by means of ultrasound and then being sucked out. The above damages the human body as other neighboring tissue cells, such as blood cells, which are also disrupted. A surgical device is therefore disclosed which comprises a liquid jet device with a particular operating hand piece (1). The operating hand piece (1) comprises a capillary (3) for the emerging liquid jet and a capillary (4) for sucking out the separated tissue cells. The emerging liquid jet is formed in cross-section so as to give a sloughing effect.

The invention relates to a surgical device for removal of tissue cells of a biological structure according to the preamble of claim 1. Such instruments are used in hospitals performing surgery.

The cell is the basic unit of the living material and comprises essentially one or several cell nuclei, which are embedded in cytoplasm and surrounded by a cell membrane. Several cells combined form a tissue in the most various kind.

There are frequently reasons to remove such tissue cells from a biological structure such as for example the human body. Excessive fat tissue from all possible body parts has frequently to be removed for health reasons or for cosmetic reasons. Such a removal is performed either in an open surgical procedure or by an ultrasound treatment. In general the ultrasound treatment is applied in practical situations, wherein an ultrasound probe and a suction cannula are inserted into the corresponding fat tissue. All bordering fat cells are destroyed and are removed through the suction cannula with the aid of the ultrasound waves exiting in all directions.

This method is associated with essential disadvantages. Naturally not only the corresponding fat cells are destroyed therewith but also all other tissue cells disposed in the operating reach, such as for example blood cells. This damages the human body and renders therefore also the healing process more complicated and therefore lengthens the healing process. A further disadvantage results therefrom that such ultrasound probes radiate in all of directions and therefore cannot be employed directed to a target. For example it is not possible to process border regions between the fat tissue to be suctioned off and a neighboring healthy tissue, without that the healthy tissue becomes damaged.

It is also part of the medical practice to remove vital tissue cells from a biological structure, for example the liver, in order to propagate the tissue cells outside of the human body by way of division and in order to feed in the thereby propagated tissue cells later on again to the corresponding organ of a human body. The removal of such vital tissue cells is performed either in an open surgical procedure or, as is usually the case, endoscopically, by mechanically separating a small group of tissue cells and by removing them. Here again the disadvantage occurs that healthy tissue cells are interfered with and are damaged. This damages and burdens again the human organism.

Therefore the invention is based on the object to develop a surgical device for removal of tissue cells from a biological structure which allows to separate and remove tissue cells without being damaged.

This object is resolved by the characterizing features of claim 1. Further embodiments results from the subclaims 2 through 8. The invention eliminates the recited disadvantages of the state of the art. The particular advantage of the invention comprises that neither the tissue cells to be removed nor the neighboring and remaining tissue cells are damaged during removal. This allows on the one hand the further use of the removed tissue cells for example in connection with a propagation of the tissue cells performed outside of the human body or of the animal body. This kind of removal, however, takes also care of all healthy tissue cells remaining in the body, since only the desired tissue cells are targeted and removed. It is advantageous for this purpose to form the liquid jet like a peeling knife or like a scraper. This occurs for example by a flat liquid jet or by several individual point shaped individual jets, which individual jets are disposed on a straight line or also along a curved line and this way form in combination again a closed and stretched liquid jet.

However it is also advantageous for several possibilities of application to employ only one point shaped liquid jet or only a slightly flattened liquid jet and to swivel this liquid jet with the aid of technical motion elements or also only by the hand movements of the operator on a line like a pendulum over a certain region. This furnishes again the effect of a flat liquid jet.

It is also advantageous if the more or less flat liquid jet extends perpendicular under a possibly right angle relative to the suction openings disposed on an axial line, because this construction furnishes the shortest transport path of the removed tissue cells between the peeling edge of the liquid jet and the suction bore holes.

The invention is to be explained in more detail by way of an embodiment example.

There is shown in:

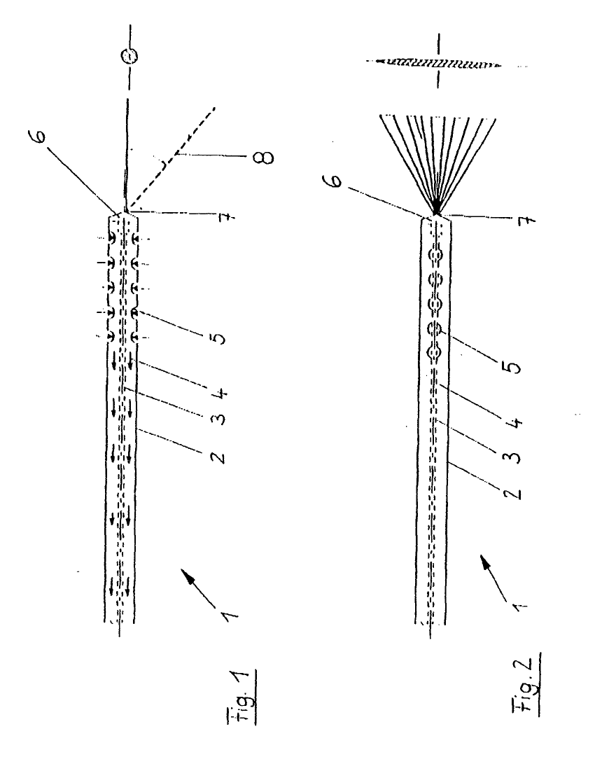

FIG. 1: the operational hand piece of the surgical device with a round liquid jet, and

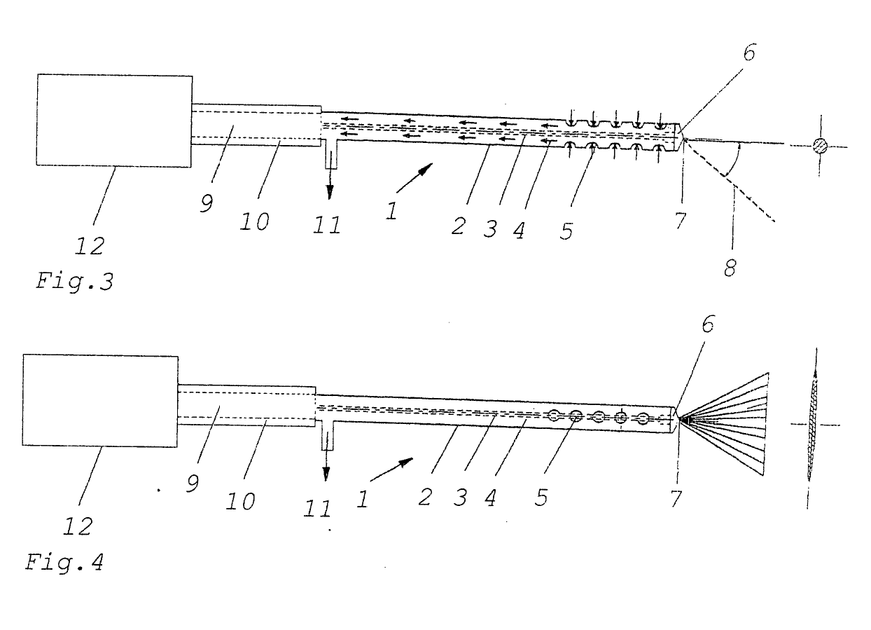

FIG. 2: the operational hand piece with a flat liquid jet.

The surgical device for removal of vital tissue cells from a biological structure corresponds in its construction substantially to a liquid jet device for separating of a biological structure. Such a separating device is a generally known and does not have to be shown here separately. For example a corresponding separating device is described in the European patent document EP 0551920 B 1.

The surgical device for removal of tissue cells from a biological structure therefore comprises essentially a pressure generator, a piston cylinder unit and a special operational hand piece 1. A cartridge is inserted shape matchingly into the cylinder chamber of the piston cylinder unit, wherein the cartridge is filled with a sterile liquid and is connected to the operating hand piece 1. During operation the pressure medium of the pressure generator burdens the cartridge, whereby the sterile liquid is pressed out of the cartridge and is transported to the operating hand piece 1, where the sterile liquid exits under pressure in the shape of a fine liquid jet. All apparatus units of this high pressure liquid jet device are constructed such and are tuned to each other such that on the one hand a sterile chain for the liquid is assured throughout and on the other hand the exiting liquid jet is capable to work on different biological structures in an intelligent fashion and with highest precision.

According to FIGS. 1 and 2, the special operating hand piece 1 comprises a handle piece not shown for the operator and a cannula 2 for insertion into the biological structure. A capillary 3 for the exiting liquid jet and a capillary 4 for suctioning off of separated tissue cells and of the used liquid are formed inside of the cannula 2. Preferably here the capillary 3 for supplying with liquid is disposed inside and coaxial to the cannula 2 and the capillary 4 for suctioning off of the tissue cells is formed as an anullar channel surrounding the inside disposed capillary 2. Alternatively, the two capillaries 3 and 4 of course can also be disposed separately next to each other in the cannula 2.

The capillary 4 for suctioning off of the tissue cells is furnished with several radial suctioning openings 5 at the distal end of the cannula 2, wherein the several radial suctioning openings 5 are disposed on two oppositely disposed axial lines at equal distances relative to each other. The diameter of the radial suctioning openings 5 are selected such that on the one hand the buildup of a suction force and on the other hand a passing off the separated tissue cells is made possible.

The capillary 3 for the exiting liquid jet also joins to a nozzle piece 6 at the distal end of the cannula 2, wherein the nozzle piece 6 is rigidly connected to the cannula 2. Preferably the nozzle piece 6 is screwed into the cannula 2. The nozzle piece 6 has an axially disposed nozzle opening 7, wherein the nozzle openings 7 is formed in a particular way for the most different fields of use.

For example the nozzle opening 7 of the nozzle piece 6 is formed flat according to FIG. 2 in the kind that an exiting liquid jet results with a heavily flattened cross-section for a removal of fat tissue cells. Here this flat liquid jet is directed such that each of the two rows of radial suction openings 5 is disposed at a right angle to one of the two flattened long sides of the liquid cross-section.

In contrast, the nozzle opening 7 of the nozzle piece 6 is formed round or nearly round according to FIG. 1 in the kind that the liquid jet exits with a round cross-section, with a round cross-section with a slight flattening, or with an oval cross-section for example for the removal of vital tissue cells from the liver. Here again the radial suction openings 5 are disposed about at a right angle to the flattenings present in the liquid jet.

The dashed exit line 8 is to indicate that the exiting liquid jet can also exit under a predetermined angle relative to the middle axis of the cannula 2 based on a corresponding shape and arrangement of the nozzle opening 7 in the nozzle piece 6.

Initially, a minimum surgical cut is performed in the abdominal wall of the patient for removal of fat tissue cells for example based on cosmetic reasons and then the cannula 2 is applied at this opening with the liquid jet already exiting under pressure. Here the opening of the abdominal wall is pressed apart so far based on the pressure of the exiting liquid jet that the cannula 2 of the operating hand piece can be easily slid under the abdominal wall up to the region of the fatty tissue. The operator can identify by touch the position of the cannula 2 and the direction and the position of the flat liquid jet with the free hand of the operator at the pressure outside of the abdominal wall derived from the liquid jet and the operator can correct or change the position of the cannula 2 and the direction and the position of the flat liquid jet with the guiding hand. The pressure of the liquid jet is automatically controlled locally such that the flat liquid jet exits with such a force, where the force does not destroy the fat tissue cells but only displaces the fat tissue cells out of the region of the jet. Neighboring fat cells are shifted thereby and lift off from each other such that free spaces are formed between the fat cells, wherein the liquid jet penetrates into the free spaces and presses apart there the still present seam positions of the neighboring cell membranes. The fat cells are peeled off from neighboring fat cells in this manner without being damaged. These effects can be further enhanced by pulsating and moving the liquid jet. The fat cell aggregates lifted off in this fashion are in the following sucked off and the separately collected through the radial suction openings and the capillary 4.

The cannula 2 off the operating hand piece 1 is equipped only with such a nozzle piece 6, where the nozzle piece 6 produces a round or an approximately round and point shaped liquid jet and a smaller pressure at the liquid jet is set at the surgical device for a removal of tissue cells for example of the liver. The cannula 2 and the pressure setting is performed in the same kind and fashion as is done during removal of fat cells. Depending on the requirements, the point shaped liquid jet is moved sideways along a line in order to increase the peeling effect. The individual or small cell aggregates are again suctioned off and are then delivered to a corresponding station for the purpose of cell propagation, multiplication and reproduction.

### List of Reference Numerals

1. operating hand piece

2. cannula

3. capillary for supplying with liquid

4. capillary for suctioning off of separated tissue cells

5. radial suction opening

6. nozzle piece

7. nozzle opening

8. dashed exit line

1. Surgical device for removal of tissue cells from a biological structure, characterized in that the surgical device is a liquid jet device comprising a pressure generator a storage container for the liquid to be placed under pressure by the pressure generator and an operating hand piece (1) for the exit of the liquid subjected to pressure, wherein the operating hand piece (1) comprises a capillary (3) for the exiting liquid jet and a capillary (4) for the suctioning off of separated tissue cells and wherein the exiting liquid jet is formed such in its cross-section that a peeling effect occurs.

2. Surgical device according to claim 1, characterized in that the cross-section of the exiting liquid jet is formed nonround.

3. Surgical device according to claim 2, characterized in that the cross-section of the exiting liquid jet is formed flat.

4. Surgical device according to claim 3, characterized in that the cross-section of the exiting liquid jet is formed by several individual jets, wherein the individual jets have a round cross-section and are disposed on the common straight or curved line.

5. Surgical device according to claim 1, characterized in that the exiting liquid jet exhibits a round cross-section and is deflected sideways to a limiting extent by motion elements.

6. Surgical device according to claim 1, characterized in that the exiting liquid jet is directed at a predetermined exit angle relative to the capillary (3).

7. Surgical device according to claim 1, characterized in that the capillary (4) is formed as an anullar channel within the operating hand piece (1) for the suctioning off of separated tissue cells, and wherein the anullar channel is furnished with the radial suction openings (5) disposed at the circumference.

8. Surgical device according to claim 7, characterized in that the suction openings (5) are disposed on two oppositely disposed axial lines and are directed at a right angle to the flat liquid jet.
